(19) United States
(12) Patent Application Publication
(10) Pub. No.: US 2024/0376481 A1
(43) Pub. Date: Nov. 14, 2024

(54) COMPOSITIONS AND METHODS FOR ENHANCING AND EXPANDING INFECTION INDUCED IMMUNITY

(71) Applicant: AIM ImmunoTech Inc., Ocala, FL (US)

(72) Inventors: Thomas K. EQUELS, Ocala, FL (US); David R. STRAYER, Bryn Mawr, PA (US)

(73) Assignee: AIM ImmunoTech Inc., Ocala, FL (US)

(21) Appl. No.: 18/692,244

(22) PCT Filed: Sep. 26, 2022
(86) PCT No.: PCT/US2022/077034

§ 371 Date: Mar. 14, 2024

Related U.S. Application Data
(60) Provisional application No. 63/248,232, filed on Sep. 24, 2021.

(51) Int. Cl. C12N 15/117; A61P 31/14; A61P 31/16

(52) U.S. Cl. CPC C12N 15/117; A61P 31/14; A61P 31/16; C12N 2310/17; C12N 2320/32

(57) ABSTRACT

Methods and compositions are provided for the treatment of virus infections in a subject which can be a human subject. The methods include administering one or more doses of a composition comprising therapeutic double-stranded RNA (tdsRNA) to a subject after active viral replication in the nasal passages of the subject. Following administration, the tdsRNA induces an enhanced and cross protective immune response to the virus or a broad-based immune response to the virus and variants thereof.

### BACKGROUND

The tremendous impact of viral infections on public health is widely recognized. Since viruses, and especially RNA viruses, have the ability to mutate rapidly, vaccines may have a limited ability to protect a patient from viral variants. For example, vaccines against the influenza virus are seasonal and are only effective for about one year, necessitating continuous monitoring for new antigenic variants, scaling up production of new vaccines to protect against next season's dominant influenza strains, and annual influenza vaccination programs. Further, seasonal flu vaccines are not optimal because the dominant influenza strains may not have been correctly predicted or the targeted influenza variants may have already been eclipsed by another variant by the time of vaccine deployment. For this reason, many people develop influenza infections even after vaccination. The effectiveness of the seasonal influenza vaccine frequently is lower than desired, and vaccine efficacy differs, year to year, depending on the emergence of viral variants.

COVID-19 presents a similar challenge to vaccine development. The recent emergence of SARS-COV-2 and its continued ability to mutate and generate new variants have raised concerns similar to those seen with influenza. That is, the ability of SARS-COV-2 to mutate may outpace the speed of vaccine development. The currently available vaccines, developed against the first SARS-COV-2 isolates, are demonstrating reduced effectiveness against some variants of SARS-COV-2, such as the delta variant. It is feared that the emergence of additional new variants of SARS-COV-2, potentially exhibiting increased transmissibility and/or, can cause a renewed or unending pandemic.

Respiratory viruses are often anthropozoonotic infectious pathogens, which provide reservoirs of dynamic genetic pools and a source of previously unknown human pathogens commonly referred to as emerging viral pathogens. The appearance of new, highly virulent strains of pathogens is a major concern for public health especially because it is impossible to predict the moment of such appearance of new viral antigens.

It is postulated that a nasal viral infection, with repeated or prolonged exposure to viral antigen, can promote the diversification of the neutralizing antibody response and maturation of the cytotoxic T-cell response, thereby targeting variable epitopes and evasion by variants of the “legacy” immunity induced by prior vaccination. It is observed that prolonged nasal viral infection can cause both (1) a primary local immune response against the virus and (2) a more general (systemic) immune response against the virus. Therefore, it is postulated that the breadth of neutralization of a patient's antibodies is contingent upon antigen persistence caused by an actual infection, which, in turn, fosters the progressive accumulation of somatic mutations and affinity maturation in the antibodies. While this is beneficial, developing a broad immune response against variants by intentional exposure to them as live viruses is undesirable with potential, unintended consequences that could be harmful.

The worldwide response to the serious threat of COVID-19 to public health is illustrative of conventional strategies for combatting infectious diseases. Antiviral drugs and vaccines are reactive against a specific virus and their effectiveness is short-lived because the virus uses its high mutation rate and rapid multiplication to evade the drug's inhibiting of the biological processes that are required for the virus' replication and the vaccine's directing the immune system to recognize the virus' antigens. Paradoxically, the initial success of drugs and vaccines to fight the infection also selects for viral variants that have altered how they replicate and lost the antigens that mark them as foreign.

There is a need for a paradigm-breaking strategy that can be implemented quickly and enhances the body's natural defenses to infection. We have selected respiratory viruses to illustrate the usefulness of our invention, which avoids the annual need to make new vaccines and to develop herd immunity in the population, many of whom are not at risk for serious illness but can spread the infection to those who have comorbidities and find it difficult to fight off infections. Our invention enhances the body's natural defenses against infection, does not need to be reengineered for succeeding waves of viral variants, and focuses on the sites where the virus initially invades the body.

### SUMMARY

This disclosure provides a safer and more effective method for inducing a broad response to optimize protective immunity against viral infection by providing an early-stage intranasal therapy to naturally exposed or early-stage infected subjects, which then confers at least (1) enhanced intranasal local immunity, (2) enhanced systemic immunity, (3) enhanced cross-reactivity to viral variants, (4) enhanced cross-protection from viral variants, (5) enhanced mucosal immunity to viral variants, or any combination thereof. The benefits of the disclosed compositions and methods provide the basis to manage current virus infections and future virus infections are discussed further below.

In one embodiment, the methods and compositions allow a safer but more prolonged nasal and/or mucosal exposure to viral antigens but without the detriment of a regular viral infection due to the immediate antiviral properties of tdsRNA. The nasal exposure to the viral antigens and replicating virus (at least partially suppressed by tdsRNA), promotes an increased epitope spreading, increased cross-reactivity of the antibodies, increased cross-protection, and increased mucosal immune response. The term “increased” may refer to a relative increase compared to a second subject (e.g., person) not administered tdsRNA or administered a placebo. The effects on an individual subject may be inferred by comparing a treated group of subjects to an untreated or a placebo group.

Increased epitope spreading refers to the diversification of epitope specificity from the initial focused, dominant epitope-specific immune response, directed against a self or foreign protein, to subdominant and/or cryptic epitopes on that protein (intramolecular spreading) or other proteins (intermolecular spreading). Some of the other proteins may be expressed on a variant of the virus. Thus, epitope spreading allows a subject to have immunity to virus variants that may evolve in the future.

Increased cross-reactivity between antigens occurs when an antibody directed against one specific antigen is successful in binding with another, different antigen. The two antigens in question have similar three-dimensional structural regions, known as epitopes, which allow the antibody against one antigen to recognize a second antigen as being similar enough structurally. Cross-reactivity may be robust among antigens of similar phylogeny such as antigens from different but related viruses—such as viruses that are variants of each other or from the same virus family. Cross-reactivity is increased by the methods and compositions of the disclosure because of the longer exposure of the nasal mucosa to the virus.

Cross protection is the phenomenon which occurs when one isolate of a virus infects a subject and, later, when the subject is exposed to a second isolate, the symptoms of the second isolate infection are suppressed, delayed, or prevented.

In one embodiment, the methods of the disclosure induce, among other effects, a mucosal immune response. Mucosal surfaces of the nasal tract and respiratory tract comprise a barrier to viral infection known as epithelial cell lines which are active participants in mucosal defense. Epithelia and their associated gland produce innate defenses including mucins and antimicrobial proteins. Epithelial cells are triggered by the presence of dangerous/foreign microbial components through Toll-like receptors (TLRs) such as TLR3 and send the cytokine and chemokine signals, including interferons, to mucous membrane-associated APCs, such as dendritic cells (DCs) and macrophages, to trigger nonspecific/innate defenses and stimulate adaptive immune responses.

One important characteristic of mucosa is the production and secretion of antibodies, such as dimeric IgA, that are resistant to degradation in the protease-rich surroundings of mucosal surfaces. In humans, the production of IgA antibodies, sometimes as much as 1 mg/mL, are present in the mucosal surface-associated secretions. IgA facilitates the entrapment of microbes such as viruses into the mucus by avoiding direct contact of pathogens with the mucosal surface in a process known as “immune exclusion.”

The disclosed nasal delivery of tdsRNA uses the virus in combination with tdsRNA to trigger a more vigorous local microbial-specific immune response. Therefore, the virus and tdsRNA combination increases the ability of the virus to trigger an initial innate immune response and, in a longer time frame, trigger a more rigorous adaptive immune response. The innate immune response will, in turn, trigger a more rigorous mucosal immune exclusion.

The enhanced immune exclusion at the mucosal surface can have significant benefits in protecting a subject exposed to the virus. For example, the initial viral exposure may be in one mucosal surface such as around the eye. A developed immune exclusion response would prevent the virus from migrating from its initial exposure site to the respiratory mucosal surfaces, where the effects of the virus are more detrimental to the host.

Briefly restating, the compositions and methods of the disclosure have two general primary activities or benefits. The first is an augmentation of the innate immune response and the second is an augmentation of the adaptive immune response. In both cases, augmentation refers to at least an improved immune response with one or more of increased antibody concentration, increased antibody affinity to antigen, increased mucosal immunity including increase immune exclusion, increased epitope spreading, increased cross-reactivity of the antibodies, increased cross-protection, or any combination thereof. The benefits of the disclosure are realized by allowing a virus to replicate in the nasal tissue and yet attenuating the pathological effects of this replication by repeated administration of tdsRNA. The tdsRNA augments the body's natural immune defenses to ensure the viral infection is not as severe as an infection without the benefit of tdsRNA administration. The increased nasal persistence of the virus allows the development of a more robust innate and adaptive immune response with significant benefits. These benefits are described in more detail herein.

Together, the ability of the disclosed compositions and methods allows the use of the disclosure as a management strategy for emerging viral diseases. The viral disease may be a re-emerging virus or a new variant of a known virus. This management strategy is especially useful where, for example, (1) the disease is endemic with no other possibility of control; (2) the disease is from a new isolate or a new virus, (3) the disease is the re-emergence of a previously known virus, and (4) the disease is from a virus (new or re-emerging) where there is no viable vaccine available or the vaccine is in short supply or difficult to distribute.

One embodiment is directed to a method for treating active virus replication in the nasal passages of a subject, comprising administering to said subject a tdsRNA.

Another embodiment is directed to a method for treating a nasal virus infection in a subject. The method comprises administering to said subject a tdsRNA, wherein administering may be at least two nasal administrations to the nasal mucosa of the subject during viral replication in the nasal mucosa of the subject.

The nasal administrations of this disclosure provide many advantages. For example, nasal administration induces mucosal immune responses and systemic immunity and provides better protection against infectious agents compared to a regular intramuscular injection. Nasal administration of tdsRNA along with viral replication elicits potent immunoglobulin A (IgA) secretion in the respiratory tract and achieves a better systemic bioavailability and protection compared with parenteral and oral administration. Nasal delivery, in the methods of the disclosure, allows the mucosal surfaces to act as in an “immune exclusion” capacity and block pathogen (e.g., virus) entry, thus increasing the general efficacy of the vaccine. In addition, antigen uptake into the blood circulatory system by absorption through mucosa can be relatively fast. Because the nasal mucosa is rich in T cells, B cells, and plasma cells, the disclosed methods stimulate both antigen-specific systemic and mucosal adaptive immune responses in addition to the innate immune response. Resident memory T cells in the nasal mucosa, once activated by the methods of the disclosure, play a part in preventing pathogen spread to the lungs to cause a more serious infection. Secreted IgA in the nasal mucosa, also activated by the methods of the disclosure, is able to bind toxins, bacteria, or viruses and neutralize their activity and thus prevent viral entry into the nasal mucosa or into the body protecting the internal organs. All these biological processes form a first barrier of defense against a pathogen and a more long-term, durable, adaptive defense against the same. An additional benefit of nasal delivery is that it provides better patient compliance due to the needle-free delivery.

In any embodiment, the administering step may be at least, for example, 2, 3, 4, 5, 6, 7, 8, 9 or 10 administrations. The interval between administration may be, for example, 1 day, 2 days, 3 days, 4 days, 5 days, 6 days or one week. One preferred interval is every other day (once every two days). One preferred dosage regimen is 7 doses, administered one dose every two days. Administration may comprise half of the dosage amount through one nostril and the second half of the dosage amount through the other nostril or any other combination such as 0 to 100% in one nostril and the remainder in the second nostril. Administration may be continuously maintained as long as a subject is at risk of exposure or if the subject is exposed to a virus daily; for example, in his/her occupation.

In any embodiment, the nasal administration may be continued (e.g., every other day) until nasal virus protein level or nasal virus nucleic acid level is reduced by 50%, 75%, 90%, 95% or 99%. Nasal virus protein may be monitored by an antibody-based assay. Nasal virus nucleic acid may be monitored by polymerase chain reaction of virus DNA or virus RNA. Continued refers to continuation of the administration method, dosage and or frequency. For example, continuation may refer to continued administration every day, every other day, or every third day, or twice a week or any other regimen in this disclosure.

In any embodiment, the method may involve an additional step of determining if there is active virus replication in the nasal passages of the subject before the administering step. In one embodiment, the administration step is started if the subject is determined to be virus positive. Virus replication may be determined by antibody assays or PCT assays to detect proteins or nucleic acids that are indicative of virus replication. Virus replication or virus presence in the subject may be determined/monitored throughout the administering step since, in some embodiments, the administering steps may require two weeks or more in time.

In any embodiment, the method or composition may reduce a symptom or a sign of the virus infection in the subject, in a part of the subject, or in the respiratory tract of the subject. A part of the subject may refer to an organ. The organ may be, for example, the lung, the nose, the nasal pharynx, the sinus, the brain, body fluids, blood, saliva, and the like, or a part thereof.

The symptom(s) or sign(s) may include, for example, at least one selected from the group consisting of: virus protein level; virus nucleic acid level; peak viral load; time to peak viral load; duration of viral shedding; viral load area under the curve (viral AUC); virus titer; nasal virus protein level; nasal virus nucleic acid level; nasal peak viral load; nasal time to peak viral load; nasal duration of viral shedding; nasal viral load area under the curve (viral AUC); nasal virus titer; cough; runny nose; nasal congestion; sore throat; headache; body aches and pains; fever; chills; fatigue; rhinorrhea; cough; and malaise. The reduction may be a reduction compared to a second subject not administered tdsRNA or administered a placebo. In any embodiment, the reduction may be determined by quantitative RT-PCR analysis of a nasal swab, a nasal wash, a body fluid, or salivary gland secretion from the subject.

In any embodiment, the administering of tdsRNA step may be started immediately after, within one day, within 2 days, within 3 days, within 4 days or within a week of (1) exposure to the virus; or (2) onset of a symptom of virus infection; or (3) a positive test for the virus.

In any embodiment, the method or composition may induce a protective immune response in the subject against the virus or a second virus. The protective immune response may be, for example, an enhanced innate immune response, an enhanced adaptive immune response, or an enhanced mucosal immune response.

In any embodiment, the method or composition may induce at least one selected from the group consisting of: enhanced cross protection; enhanced epitope spreading; enhanced cross reactivity; and enhanced mucosal immunity.

In any embodiment, the second virus may be a variant of the virus or virus with common antigenic immune epitopes. Using SARS-COV-2 as an example, the original infection may be caused by the alpha variant of SARS-COV-2 while the broad based immune response may provide protection (prevent or attenuate one or more symptoms of) against one of more of the other variants such as the Beta, Gamma, Delta, Eta, Iota, Kappa, Lambda or Mu variants while developing more complete and robust immunity against the variant. Using influenza as an example, if the initial infection is caused by one strain of influenza, the broad based immune response may provide protection (prevent or attenuate viral replication against another strain of influenza (e.g., H1N1, H3N2, H5N1, H5, H7, H7N9, H5N6, H10N8, H9N2, and H6NI or currently unrecognized H and N variants beyond the current 16 H and 9 N variants).

In any embodiment, the method may produce a broad-based response in the subject. The broad-based immune response may be a broad-based immune response to a second virus. As stated above, the second virus may be a different virus with common linear or conformational epitopes or may be a variant of the virus. The method or the broad-based immune response enhanced by the method may reduce a characteristic of the second virus infection in the subject. These infectivity characteristics of the second virus may be one or more selected from the group consisting of second virus protein level; second virus nucleic acid level; peak second virus viral load; time to peak second virus viral load; duration of second virus viral shedding; second virus viral load area under the curve (viral AUC); and second virus titer.

In any embodiment, a symptom or sign of a second virus infection may be at least one selected from the group consisting of: second virus protein level; second virus nucleic acid level; peak second viral load; time to peak second viral load; duration of second viral shedding; second viral load area under the curve (viral AUC); second virus titer; cough; runny nose; nasal congestion; sore throat; headache; body aches and pains; fever; chills; fatigue; rhinorrhea; cough; and malaise.

In any embodiment, the subject is preferably a mammal, more preferably a human. Other subjects may be rodent, primate, rat, mouse, horse, donkey, sheep, pig, cow, deer, goat, dog, cat, rabbit, bat, ferret, or any animal mentioned in this disclosure.

In any embodiment, the virus (i.e., the first virus) or the second virus independently may be at least one selected from the group consisting of: influenza virus; adenovirus; herpes virus; rhinovirus; respiratory syncytial virus (RSV); Influenza A; Influenza B; H1N1 influenza; H3N2 influenza; H7N9 influenza; H5N6 influenza; H1ON8 influenza; H9N2 influenza; H6N1 influenza; Human coronavirus 229E (HCoV-229E); Human coronavirus OC43 (HCoV-OC43); Human coronavirus NL63 (HCoV-NL63, New Haven coronavirus); Human coronavirus HKU1; coronavirus; Ebola Virus; West Niles Virus; Zika Virus; H5 influenza; H7 influenza; H5N1 influenza; West Niles Virus; Zika Virus; SARS-COV; SARS-COV-1; SARS-CoV-2; MERS-COV; HCoV-EMC; and a variant thereof.

The tdsRNA, in any part of this disclosure, refers to a double-stranded RNA with a formula that is at least one selected from the group consisting of

rIₙ·r(CₓU)ₙ (formula 1);

rIₙ·r(CₓG)ₙ (formula 2);

rAₙ·rUₙ (formula 3);

rIₙ·ᵣCₙ (formula 4);

and

rugged dsRNA (formula 5).

In any of these formulas, x can be at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

The tdsRNA, in any part of this disclosure, may have a size distribution where at least 90 wt % of the tdsRNA is larger than a size selected from the group consisting of: 40 basepairs; 50 basepairs; 60 basepairs; 70 basepairs; 80 basepairs; and 380 basepairs. Independently, the tdsRNA, in any part of this disclosure, may have a size distribution where at least 90 wt % of the tdsRNA is smaller than a size selected from the group consisting of: 50,000 basepairs; 10,000 basepairs; 9000 basepairs; 8000 basepairs; 7000 basepairs; and 450 basepairs. In any part of this disclosure, the variable n (e.g., in the formulas) may have a value selected from the group consisting of: 40 to 50,000; 40 to 40,000; 50 to 10,000; 60 to 9000; 70 to 8000; 80 to 7000; and 380 to 450.

In one embodiment, the tdsRNA may have the following characteristic such as: (1) n (in the formulas 1-5 for tdsRNA) may be from 40 to 40,000; (2) the tdsRNA has about 4 to about 4000 helical turns of duplexed RNA strands; or (3) the tdsRNA may have a molecular weight selected from the group consisting of: 2 kDa to 30,000 kDa; 25 kDa to 2500 kDa; and 250 kDa to 320 kDa.

In a preferred embodiment, the tdsRNA may comprise or consist of rIn·r(C₁₁₋₁₄U)ₙ; and rugged dsRNA.

In any embodiment, the rugged dsRNA may have (1) a single strand comprised of r(C₄₋₂₉U)ₙ, r(C₁₁₋₁₄U)ₙ, or r(C₁₂U)ₙ; (2) an opposite strand comprised of r(I); (3) wherein the single strand and the opposite strand do not base pair the position of the uracil base, and (4) wherein the single strand and the opposite strand are partially hybridized.

In any embodiment, the rugged dsRNA may comprise (1) a molecular weight of about 250 kDa to 500 kDa; (2) a structure where each strand of the rugged dsRNA is from about 400 to 800 basepairs in length; or (3) 30 to 100 or 30-60 helical turns of duplexed RNA.

In any embodiment, the tdsRNA may comprise rugged dsRNA or consist of rugged dsRNA. The Rugged dsRNA may be resistant to denaturation under conditions that are able to separate hybridized poly(riboinosinic acid) and poly(ribocytosinic acid) strands (rIₙ·rCₙ).

In any embodiment, the rugged dsRNA may be an isolated double-stranded ribonucleic acid (dsRNA) enzymatically active under thermal stress comprising: each strand with a molecular weight of about 250 KDa to about 500 KDa, 400-800 basepairs, or 30 to 60 helical turns of duplex RNA; a single strand comprised of poly(ribocytosinic₄₋₂₉ uracilic acid) and an opposite strand comprised of poly(riboinosinic acid); wherein the two strands do not base pair the position of the uracil base; wherein the two strands base pair the position of the cytosine base; and wherein said strands are partially hybridized.

In any embodiment, the tdsRNA may comprise 0.1-12 mol % rugged dsRNA. In a preferred embodiment, the tdsRNA comprises 0.1-5 mol % rugged dsRNA.

In any embodiment, the tdsRNA may be in a composition with at least one pharmaceutically acceptable carrier. The pharmaceutically acceptable carrier may be, for example, water (including RNase fee water), buffer, or Phosphate Buffered Saline (PBS). PBS may have a formulation of 0.15 M NaCl, 0.01 M Na₃PO₃, 0.001 M MgCl₂ or 0.138 M NaCl; 0.0027 M KCl. The pH of PBS may be pH 7.4.

In any embodiment, the tdsRNA may be a stabilized tdsRNA. The stabilized tdsRNA may be a dried tdsRNA such as, for example, a lyophilized tdsRNA. The stabilized tdsRNA may be recovered-that is, the stabilized tdsRNA may be reconstituted into a liquid solution. In one embodiment, the stabilized tdsRNA, especially in the dried form including the lyophilized form, may be used directly in the dried state. In one aspect, the stabilized tdsRNA may be an alcohol (e.g., ethanol) precipitate of tdsRNA. In one aspect, the alcohol precipitate of dsRNA may be used directly. Optionally, before the administering step or before use, the stabilized tdsRNA may be dissolved with the appropriate diluent such as phosphate buffered saline. Alternatively, the tdsRNA may be dried in PBS and resuspension is made with water of the original volume causing the tdsRNA and the PBS to be reconstituted. tdsRNA alcohol precipitates may be removed from the alcohol by centrifugation and resuspended. If needed, in any embodiment, the buffer can be changed using a commercially available column for buffer exchange.

When the tdsRNA is in a dried form including in a lyophilized form, the ddsRNA may be substantially free of moisture. Preferably, the dried tdsRNA has a moisture content selected from the group consisting of: less than 1%, less than 3%, less than 5%; between 5-10%; between 10-15%; between 15-20%; and between 20-25%.

In any embodiment, the tdsRNA may be complexed with a stabilizing polymer. The stabilizing polymer may be at least one selected from the group consisting of: polylysine; polylysine and carboxymethylcellulose; polyarginine; polyarginine and carboxymethylcellulose.

In one embodiment, the tdsRNAs of the disclosure are provided to the subject having a risk of exposure to one or more viruses disclosed herein. In one embodiment, tdsRNA of the invention are administered to a subject every two days for 1, 2, 3, 4, 5, 6 or 7 administrations. That is, for example, 7 administration would be administered on days 1, 3, 5, 7, 9, 11 and 13. In another embodiment, the administration is continuous. For example, one administration every other day for a period of 2 weeks, one month, two months, three months, to one year or more.

tdsRNA compositions of the disclosure are typically prepared as sterile, aqueous PBS solutions. These solutions are stable under conditions of manufacture and storage. In some aspects, the tdsRNA may be prepared as a stabilized tdsRNA which can be a dry form of tdsRNA. The dried tdsRNA may be packaged with a preferred pharmaceutically acceptable carrier which can be, for example, water or PBS. The dried form of tdsRNA can be used directly, as a powder for nasal administration. Alternatively, before use, the dried tdsRNA may be mixed with the pharmaceutically acceptable carrier to constitute a formulation of tdsRNA for administration by any of the methods disclosed. One preferred administration method is intranasal administration.

Treatment progress for subjects receiving tdsRNA can be monitored and additional administrations provided. For example, if virus is detected in the nasal cavity, additional tdsRNA may be administered. In addition to a reduction in viral infection symptoms, viral infection progress can be monitored by assaying for viral infection markers such as virus antigen levels. Broad-based response in a patient may be determined by a subject's antibody titers. Based on an individual patients' progress, additional tdsRNA injections can be performed in accordance with the present invention. In some embodiments, the tdsRNA are performed in an indefinite series, for example, to protect a health care worker with constant exposure to a virus.

Embodiments described in this disclosure can be combined with other conventional therapies for the target disease state or condition. For example, intravenous tdsRNA has an antiviral effect. If, in a particular case, a viral infection is not diminishing under nasal tdsRNA treatment alone, intravenous tdsRNA treatment may be applied in parallel with the methods of this disclosure to reduce the viral load on the patient. As an example, if, after two administrations of tdsRNA delivered once every two days, the viral load or viral infection symptoms is increasing to an unacceptable level, tdsRNA may be administered intravenously to a subject in parallel with the disclosed method. In this example, the virus may be any virus of this disclosure including an influenza virus or a SARS-COV-2 virus.

In any embodiment, administering and administration may be intranasal administration, inhalation administration, systemic administration, or topical administration. Other administrating methods include any method discussed in this disclosure. The administration may be performed by a delivery system or medical device such as a nasal spray. Examples of such devices include, for example, a nebulizer; a sprayer; a nasal pump; a squeeze bottle; a nasal spray; a syringe sprayer or plunger sprayer (a syringe providing pressure to an attached sprayer or nozzle); a swab; a pipette; a nasal irrigation device; or a nasal rinse.

One preferred route of administration is nasal administration. In nasal administration, the preferred dosages are 0.1 μg to 1,200 μg; 0.1 to 25 μg; 25 μg to 50 μg; 50 μg to 100 μg; 100 μg to 200 μg; 200 μg to 400 μg; 400 μg to 800 μg; or 800 μg to 1,250 μg 1250 μg to 1500 μg; 1500 μg to 2000 μg; or 2000 μg to 2500 μg; or. For example, intranasal dosages may be 25 μg; 50 μg; 125 μg; 250 μg; 500 μg; 1,000 μg; 1,250 μg; 1500 μg; 2000 μg; or 2500 μg.

In any embodiment, the tdsRNA may be administered at a frequency selected from the group consisting of: one dose per day, one dose every 2 days, one dose every 3 days, one dose every 4 days, one dose every 5 days, one dose a week, two doses a week, three doses a week, one dose every two weeks, one dose every 3 weeks, one dose every 4 weeks, and one dose a month.

In a preferred embodiment, nasal administration may be one administration every 2 days. During administration, the dosage may split into two halves and one half being delivered to one nostril and the second half delivered to the second nostril.

One preferred embodiment is directed to a method for treating a viral infection in a subject comprising nasally administering tdsRNA to the subject during nasal viral replication. Nasally administering is at least two or more nasal administrations during nasal virus replication in the subject. For example, there can be 2, 3, 4, 5, 6, 7, 8, 9, 10, 11 or 12 administrations given every other day. Treating may be at least one selected from the group consisting of: reducing nasal virus protein level; reducing nasal virus nucleic acid level; reducing nasal peak viral load; reducing nasal time to peak viral load; reducing nasal duration of viral shedding; reducing nasal viral load area under the curve (viral AUC); and reducing nasal virus titer. The method may have an effect of enhancing cross-protection; enhancing epitope spreading; enhancing cross-reactivity; or enhancing mucosal immunity; in the subject. In the method, or in any method, nasal administration may be continued at least every other day until nasal virus protein level or nasal virus nucleic acid level is reduced by at least 50%, at least 60%, at least 70%, at least 80%, at least 90%, at least 95%, at least 98% or at least 99%. The reduction may be relative to an initial level before treatment or a peak level of virus. Another feature is that the method may (1) enhance cross-protection against a second virus; (2) enhance epitope spreading to epitopes in a second virus; (3) enhance cross-reactivity against a second virus; or (4) enhance mucosal immunity against a second virus.

Another embodiment is directed to a composition for treating a virus infection, relieving a symptom thereof, or for developing a broad-based immune response in a subject comprising a tdsRNA. The tdsRNA may be any tdsRNA or tdsRNA combination described in this disclosure including the tdsRNA described for the methods of the disclosure. Another embodiment is directed to a delivery system comprising any of the tdsRNA containing compositions of this disclosure.

In any embodiment, the delivery system may be selected from the group consisting of a nebulizer; a sprayer; a nasal pump; a squeeze bottle; a nasal spray; a syringe sprayer or plunger sprayer (a syringe providing pressure to an attached sprayer or nozzle); a swab; a pipette; a nasal irrigation device; a nasal rinse; a peripheral venous catheter; a needle, a tube, a line, a central venous catheter, a peripherally inserted central catheter, a tunneled catheter, or an implanted port.

For example, the tdsRNA may be a stabilized tdsRNA; a dried tdsRNA; a stabilized and recovered tdsRNA; an alcohol precipitate of tdsRNA. The tdsRNA may be a dried tdsRNA substantially free of moisture, preferably with a moisture content selected from the group consisting of: less than 1%, less than 3%, less than 5%; between 5-10%, between 10-15%, between 15-20%, and between 20-25%

Another embodiment is directed to a medicament comprising the tdsRNA this disclosure. The tdsRNA, in any embodiment, would include any tdsRNA described in the methods of this disclosure.

Another embodiment is directed to a kit comprising the dried tdsRNA of this disclosure with a recovery fluid. For example, the dried tdsRNA and the recovery fluid are in two different compartments in a single container. The two different compartments may be separated by a liquid impermeable separator. Further, the liquid impermeable separator may be removable or breakable without breaking the container.

### DETAILED DESCRIPTION

### 1. Overview

Respiratory viruses and their variants have been taking a heavy toll on global health causing a long-felt need for developing a broad ranged immunity to viral respiratory diseases. Even before the emergence of COVID-19, it was not uncommon for influenza variants to cause 60,000 deaths in the United States in one year. Recently, virus variants, such as variants of influenza and the Delta (B.1.617.2) Variant of Concern (VOC) of SARS-COV-2 have been taking a heavy toll on global health.

To date, the long-term vaccine protection against viral variants is still lower than desired. Recent studies show that flu vaccination reduces the risk of flu illness by between 40% and 60% among the overall population during seasons when most circulating flu viruses are well-matched to those used to make flu vaccines. Vaccine Effectiveness (VE) estimates from 2004-2015 found an average VE of 33% (CI=26%-39%) against illnesses caused by H3N2 viruses, compared with 61% (CI=57%-65%) against H1N1 and 54% (CI=46%-61%) against influenza B virus illnesses. Also, vaccine effectiveness studies have shown that flu vaccines have better protection against slower mutating flu viruses (e.g., influenza B or influenza A(H1N1) viruses and less protection against faster mutating flu viruses (e.g., influenza A(H3N2). At present, there is still no evidence-based, long-term correlate of protection for SARS-COV-2. While the short-term effectiveness of a few COVID-19 vaccine was demonstrated, reports of decreasing immunity in terms of decreasing antibody are beginning to surface for all COVID-19 vaccines. There is a fear that, should SARS-COV-2 become faster in its mutation rate, it would become more resistant to standard vaccine-induced immunity against COVID-19; particularly in patients that are SARS-COV-2-naïve vaccinees.

This disclosure seeks to address a long-felt need for compositions and methods for preventing or attenuating a virus infection. One method of achieving this goal is to develop a method to induce a broad-based immune response against a virus in the nasal cavity of a subject. The broad-based immune response would treat, prevent, or at least reduce the symptoms of a second virus which may be a variant of the initial virus. Furthermore, this broad based immune response would be particularly beneficial to patients that are SARS-COV-2-naïve vaccinees, which is a population that is particularly susceptible to breakthrough infections.

The disclosed methods and compositions are suitable for use with any viruses that infects a mammalian host, preferably a human host, through the air via respiratory droplets or aerosols. One example of such a virus would be the influenza virus which is a major cause of disease in humans and a source of significant morbidity and mortality worldwide. Because variants of influenza virus appear frequently, annual vaccination is the primary strategy for preventing infections. On occasion, an influenza pandemic can occur when a new influenza virus emerges for which people have little or no immunity. In the past century, at least three influenza A strain pandemic outbreaks have caused significant human influenza-related fatalities (1918, HIN1; 1957, H2N2; 1968, H3N2). Another example of a virus of concern is SARS-COV-2 and its variants and related coronaviruses. Other respiratory viruses are currently not serious threats to public health, but variants may emerge from them having high infectiousness and virulence as exemplified by the usually mild illness caused by coronaviruses and the emergence of COVID-19. Therefore, this disclosure may also be useful to treat a subject who is at least infected with an adenovirus, a bocavirus, a coronavirus, a metapneumovirus, a parainfluenza virus, a respiratory syncytial virus, or a rhinovirus (especially those respiratory viruses who include humans in their host range or have recently acquired the ability to infect humans). For the young and the elderly whose immune systems may not adequately control infections that do not cause serious illness in healthy adults, our disclosure may enhance a frail immune response and is available to treat them early in the course of infection when the virus is initially contacting the respiratory system and its mucosal surfaces.

Because it is not possible to predict which variant of a virus, such as influenza or SARS-COV-2, will continue the current pandemic or start a new pandemic, there is a need to develop methods and compositions to address future virus emergence. The methods and compositions of this disclosure is designed to address this need. These methods and compositions would protect the host (i.e., the subject) from severe disease or death by eliciting a direct immune response, and a second more broad-based immune response. It is expected that the immune response would protect the host against a broad range of viral variants and subtypes. At present, the available vaccines rely on the induction of a neutralizing antibody response primarily against one antigen of a virus (e.g., the spike protein of SARS-COV-2) and tend to be more variant specific in their protection. Unlike current vaccines, the disclosed method seeks to develop a broad-based immune response and, at the same time, reduce the severity of a viral exposure.

In one embodiment, the disclosure provides methods and compositions (formulations) which induce a broad-based immune response in a subject. One benefit of the compositions and methods of this disclosure is that the compositions of the disclosure, when administered in a correct regimen, can reduce the severity of a viral infection and also prevent or attenuate a subsequent viral infection from the same or a variant of the virus. Embodiments of the invention provide new doubles stranded RNAs (tdsRNA), for enhanced induction of broad-based immunity, especially nasal broad-based immunity. The methods and compositions disclosed also provide a significant boost of a broad-based response in a subject.

### 2. Definitions

This disclosure relates to, inter alia, tdsRNA. tdsRNA can also be called “therapeutic dsRNA,” or “therapeutic double-stranded RNA” and these terms have the same meaning. In this section, or anywhere in this disclosure, a reference to tdsRNA would include, at least, a reference to a composition comprising tdsRNA or a medicament comprising tdsRNA. Further, any reference to tdsRNA would include at least AMPLIGEN® (rintatolimod).

“r” and “ribo” have the same meaning and refer to ribonucleic acid or the nucleotides or nucleosides that are the building block of ribonucleic acid.

RNA consists of a chain of linked units called nucleotides. This disclosure relates mostly to RNA and, therefore, unless otherwise specified, the nucleotides and bases expressed refers to the ribo form of the nucleotide or base (i.e., ribonucleotide with one or more phosphate groups). Therefore “A” refers to rA or adenine, “U” refers to rU or uracil, “C” refers to rC or cytosine, “G” refers to rG or guanine, “I” refers to rI or inosine, “rN” refers to rA, rU, rC, rG or rI. Each of these (i.e., A, U, C, G, I) may have one or more phosphate groups as discussed above depending on whether they are part of a chain (i.e., RNA) or free (nucleoside, nucleotide, etc.).

“n” is a positive number and refers to the length of ssRNA or dsRNA or to the average length of a population of ssRNA or dsRNA. “n” can be a positive integer when referring to one nucleic acid molecule or it can be any positive number when it is an average length of a population of nucleic acid molecules.

An RNA may have a ratio of nucleotides or bases. For example, r(C₁₂U)ₙ denotes a single RNA strand that has, on average 12 C bases or nucleotides for every U base or nucleotide. As another example, r(C₁₁₋₁₄U)ₙ denotes a single RNA strand that has, on average 12 C bases or nucleotides for every U base or nucleotide.

Formulas: As an example, the formula “rIₙ·r(C₁₂U)ₙ” can be expressed as riboIₙ·ribo(C₁₂U)ₙ, rIₙ·ribo(C₁₂U)ₙ, or riboIₙ·(C₁₂U)ₙ, refers to a double-stranded RNA with two strands. One strand (rIₙ) is poly ribo-inosine of n bases in length. The other strand is ssRNA of random sequence of C and U bases, the random sequence ssRNA is n bases in length, and a ratio of C bases to U bases in the random sequence ssRNA is about 12 (i.e., mean 12 C to 1 U). The terms “r” and “ribo” have the same meaning in the formulas of the disclosure. Thus, rI, ribol, r (I) and ribo(I) refer to the same chemical which is the ribose form of inosine. Similarly, rC, riboC, r(C) and ribo(C) all refer to cytidine in the ribose form which is a building block of RNA. rU, riboU, r(U) and ribo(U) all refer to Uracil in the ribose form which is a building block of RNA.

The “·” symbol indicates that one strand of the dsRNA is hybridized (hydrogen-bonded) to the second strand of the same dsRNA. Therefore, rIₙ·r(C₁₂U)ₙ is double-stranded RNA comprising two ssRNA. One ssRNA is poly(I) and the other ssRNA is poly (C₁₂U). It should be noted that while we referred to the two strands being hybridized, not 100% of the bases form base pairing as there are some bases that are mismatched. Also, because rU does not form base pairing with rI as well as rC form base paring with rI, rU provides a focus of hydrodynamic instability in rIₙ·r(C₁₂U)ₙ at the locations of the U bases.

As another example, the formula “rIₙ·r(C₁₁₋₁₄U)ₙ” refers to the same dsRNA except that a ratio of C bases to U bases one strand is about 11 to about 14. That is, the ratio can be 11, 12, 13 or 14 or any value between 11 and 14. For example, when half of the strands are r(C₁₂U)ₙ and half of the strands are r(C₁₃U)ₙ, the formula would be r(C_{12.5}U)ₙ.

The dsRNA (tdsRNA) and ssRNA of this disclosure are homopolymers (e.g., a single-stranded RNA where every base is the same) or heteropolymers (e.g., a single-stranded RNA where the bases can be different) of limited base composition. The tdsRNAs are not mRNA and are distinct from mRNA in structure. For example, the ssRNA and dsRNA are preferably missing one or all of the following: (1) 5′ cap addition, (2) polyadenylation, (3) start codon, (4) stop codon, heterogeneous protein-coding sequences, and (5) spice signals.

As used herein, the term “substantially free” is used operationally in the context of analytical testing of the material. Preferably, purified material is substantially free of one or more impurities. In a preferred embodiment, the tdsRNA of this disclosure is substantially free (e.g., more than 0% to less than 0.1%) or completely free (0%) of dI/dI dsRNA or dCdU/dCdU dsRNA. In other words, the tdsRNA is substantially free or completely free (0%) of homodimers of polymer 1 or homodimers of polymer 2. Substantially free in this context would be considered to be more than 0% but less than 1%, less than 0.5%, less than 0.2%, less than 0.1%, or less than 0.01% of a contaminant such as (1) dI/dI (polymer 1/polymer 1) dsRNA, dCdU/dCdU (polymer 2/polymer 2) dsRNA.

The terms “intranasal administration” or “intranasally,” as used herein, refer to a route of delivery of an active compound to a subject by spraying into the nose of the subject.

A particle, a droplet, or an aerosol, and the like as delivered in this disclosure may be a liquid suspension particle or a dry particle.

Active ingredients or active agents are used interchangeably and include any active ingredient or active agent described in this disclosure including, at least, tdsRNA.

The double-stranded RNAs described in this disclosure are therapeutic double-stranded RNA, abbreviated as “tdsRNA.” tdsRNA includes, at least, Rintatolimod which is a tdsRNA of the formula rIₙ·r(C₁₂U)ₙ). tdsRNA may be stored or administered in a pharmaceutically acceptable solution such as Phosphate Buffered Saline (PBS).

The tdsRNA may be a tdsRNA produced by any of the methods of this disclosure—referred to herein as the “tdsRNA Product” or “tdsRNA”-the two terms have the same meaning. tdsRNA can be represented by one or more of the formulas below in any combination:

rIₙ·r(CₓU)ₙ (formula 1)

rIₙ·r(CₓG)ₙ (formula 2)

rAₙ·rUₙ (also called polyA·polyU) (formula 3)

rIₙ·rCₙ (formula 4)

rugged dsRNA (formula 5)

Each will be discussed further below.

In some embodiments, the tdsRNA may be represented by one or more of the formulas as follows:

rIₙ·r(CₓU)ₙ (formula 1)

rIₙ·r(CₓG)ₙ (formula 2)

In any embodiment, x may be at least one selected from the group consisting of: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29 (4 to 29), 4-30 (4 to 30), 4-35 (4 to 35), 11-14 (11 to 14), 30-35 (30 to 35). Of these, x=12, and x=11-14 (x may be any value between 11 to 14) are especially preferred.

In these formulas 1 to 5, and in other formulas, where there is no subscript next to a base, the default value is “1.” For example, in the formula rIₙ·r(C₁₂U)ₙ, there is no subscript following “U,” it is understood that rIₙ·r(C₁₂U)ₙ, is the same as rIₙ·r(C₁₂U₁)ₙ and the formula is meant to convey that for the strand denoted as r(C₁₂U₁)ₙ, there are 12 rC base for every rU base. Thus, x is also a ratio of the bases of one strand of the tdsRNA. The length of the tdsRNA strand is denoted as a lowercase “n” (e.g., rIₙ·r(C₁₂U)ₙ). The subscript n is also the length of each individual single-stranded nucleic acid. Since tdsRNA is double-stranded, n is also the length of the double-stranded nucleic acid—i.e., the length of the tdsRNA. For example, rIₙ·r(C₁₂U)ₙ indicates, inter alia, a double-stranded RNA with each strand with a length of n.

In another aspect, the tdsRNA may have a formula as follows:

rAₙ·rUₙ (also called polyA·polyU) (formula 3)

rIₙ·rCₙ (formula 4)

In another aspect, the tdsRNA may be a rugged dsRNA (formula 5).

In one embodiment, tdsRNA is at least one selected from the group consisting of formula 1, formula 2, formula 3, formula 4, and formula 5. In another embodiment, tdsRNA comprises formula 1 and formula 2 only. In one preferred embodiment, tdsRNA comprises formula 1 only. In another embodiment, tdsRNA comprises formula 1 and formula 5 (rugged dsRNA) only.

In another aspect, at least 70%, at least 80%, or at least 90% of the tdsRNA may have a molecular weight of between 400,000 Daltons to 2,500,000 Daltons. Where the term percent (“%”) is used, the percent may be weight percent or molar percent.

In another aspect, the tdsRNA comprises a first ssRNA and a second ssRNA and each of these first ssRNA or second ssRNA may contain one or more strand breaks.

In another aspect, the tdsRNA has the property that greater than about 90%, greater than 95%, greater than 98%, greater than 99%, or 100% of the bases of the RNA are in a double-stranded configuration.

In any aspect, the tdsRNA may be in a therapeutic composition comprising, for example, a tdsRNA, and a pharmaceutically acceptable excipient (carrier).

One embodiment of tdsRNA is directed to rintatolimod, which is a tdsRNA of the formula rIₙ·r(C₁₂U)ₙ and which is also denoted by the trademark AMPLIGEN®.

In a preferred embodiment, the tdsRNA are of the general formula rIₙ·r(C₁₁₋₁₄, U)ₙ and are described in U.S. Pat. Nos. 4,024,222 and 4,130,641 (which are incorporated by reference herein) or synthesized according to this disclosure.

In the case where the tdsRNA is rAₙ·rUₙ, the tdsRNA may be matched (i.e., not in mismatched form).

tdsRNA (e.g., Rintatolimod) has undergone extensive clinical and preclinical testing. It has been well-tolerated in clinical trials enrolling over 1,200 patients with over 100,000 doses administered and there have been no drug-related deaths. Two placebo-controlled, randomized studies show no increase in serious adverse events compared to placebo. Favorable safety profiles have been seen for many forms of administration including intraperitoneal, intravenous, and intranasal routes of administration of tdsRNA.

tdsRNA has been shown to have a beneficial effect when administered intravenously against some coronaviruses such as SARS-COV-1 in vitro. SARS-COV-2 shares key genomic and pathogenic similarities with SARS-COV-1. Both viruses utilize the same ACE2 receptor to bind to and infect human cells. In addition, the RNA sequences of the SARS-COV-1 virus in key areas required for viral replication are almost identical to SARS-COV-2. Therefore, it was possible that Ampligen® would have similar antiviral activity against the SARS-COV-2 as it did against the SARS-COV-1. Since, at the time, there were no available mouse models for SARS-COV-2, Ampligen® was tested in vitro in a SARS-COV-2 infection model using human-derived tracheal/bronchial epithelial cells. Ampligen® decreased SARS-COV-2 infectious viral yields by 90% (Utah State University, Study Number 8520-AIM-3D-COVID-19, unpublished data on file at AIM) at clinically achievable intravenous dosage levels. A 700 mg intravenous dose of Ampligen® yields peak blood levels of 70-75 μg/ml. The EC-90 against SARS-COV-1 infectious viral load was 55 μg/ml.

In addition, using the same human-derived tracheal/bronchial epithelial cell model system, the cell cytotoxicity concentration of Ampligen® that would cause 50% cell death (CC50) was determined using the MTT assay. Ampligen® tested at 10 mg/mL was 47% cytotoxic, 4.5 mg/mL was 12% cytotoxic, and the lower concentrations (1.5 and 0.5 mg/mL) had no measurable toxicity. The data indicate that the cell cytotoxicity concentration of Ampligen® that would cause 50% cell death (CC50) is >10 mg/mL in the normal, human-derived tracheal/bronchial epithelial cell model.

Ampligen tested at 10 mg/mL was 47% cytotoxic, 4.5 mg/mL was 12% cytotoxic, and the lower concentrations (1.5 and 0.5 mg/mL) had no measurable toxicity. The data indicate that the cell cytotoxicity concentration of Ampligen that would cause 50% cell death (CC50) is >10 mg/mL in the normal, human derived tracheal/bronchial epithelial cell model (Utah State University, Study Number 8520-AIM-3D-COVID-19, unpublished data on file at AIM).

Taken together, these data demonstrate that Ampligen, applied intravenously, may be a possible prophylactic and early-onset therapeutic against COVID-19, with potential qualities as a standalone therapeutic if administered before the viral load becomes excessive.

3.1 Length of tdsRNA

The length of the tdsRNA, may be represented by bases for one strand of the tdsRNA or in basepairs for both strands for the tdsRNA. It is understood that in some embodiments that not all of the bases (e.g., U and I) are in basepaired configuration. For example, rU bases do not pair as well as rC bases to inosine.

The length of the tdsRNA may be measured by (1) bases or basepairs, (2) molecular weight which is the weight of the double-stranded tdsRNA (e.g., Daltons) or (3) turns of the double-stranded RNA. These measurements can be easily interconverted. For example, it is generally accepted that there are about 629 Daltons per base pair.

“n” represents length in units of basepair or basepairs (abbreviated as bp regardless of whether it is singular or plural) for double-stranded nucleic acid. “n” can also represent bases for single-stranded RNA. Because “bp” represents singular or plural, it is the same as “bps” which is another representation of basepairs.

The tdsRNA can have the following values for its length “n” (in bases for single strand or basepairs for double strands): 4-5000, 10-50, 10-500, 10-40,000, 40-40,000, 40-50,000, 40-500, 50-500, 100-500, 380-450, 400-430, 400-800 or a combination thereof. Expressed in molecular weight, the tdsRNA may have the following values: 30 kDa to 300 kDa, 250 kDa to 320 kDa, 270 kDa to 300 kDa or a combination thereof. Expressed in helical turns, the tdsRNA may have 4.7 to 46.7 helical turns of duplexed RNA, 30 to 38 helical turns of duplexed RNA, 32 to 36 helical turns of duplexed RNA or a combination thereof.

The length may be an average basepair, average molecular weight, or an average helical turns of duplexed RNA and can take on integer or fractional values.

3.2 Rugged DSRNA (a Form of tdsRNA)

Rugged dsRNA is a tdsRNA that is resistant to denaturation under conditions that are able to separate hybridized poly(riboinosinic acid) and poly(ribocytosinic acid) strands (that is, rIₙ·rCₙ strands). Sec, U.S. Pat. Nos. 8,722,874 and 9,315,538 (incorporated by reference) for a further description of Rugged dsRNA and exemplary methods of preparing such molecules.

In one aspect, a rugged dsRNA can be an isolated double-stranded ribonucleic acid (dsRNA) which is resistant to denaturation under conditions that are able to separate hybridized poly (riboinosinic acid) and poly (ribocytosinic acid) strands, wherein only a single strand of said isolated dsRNA comprises one or more uracil or guanine bases that are not base-paired to an opposite strand and wherein said single strand is comprised of poly(ribocytosinic₃₀₋₃suracilic acid). Further, the single strand may be partially hybridized to an opposite strand comprised of poly (riboinosinic acid). In another aspect, rugged dsRNA may be an isolated double-stranded ribonucleic acid (dsRNA) which is resistant to denaturation under conditions that are able to separate hybridized poly (riboinosinic acid) and poly (ribocytosinic acid) strands.

In another aspect, Rugged dsRNA, has at least one of the following: r(Iₙ)·r(C₄₋₂₉U)ₙ, r(Iₙ)·r(C₁₂U)ₙ, r(Iₙ)·r(C₁₁₋₁₄U)ₙ, r(Iₙ)·r(C₃₀U)ₙ, or r(Iₙ)·r(C₃₀₋₃₅U)ₙ. In another aspect, Rugged dsRNA may have a size of 4 bps to 5000 bps, 40 bps to 500 bps, 50 bps to 500 bps, 380 bps to 450 bps, 400 bps to 430 bps, 30 kDa to 300 kDa molecular weight, 250 kDa to 320 kDa molecular weight, 270 kDa to 300 kDa molecular weight, 4.7 to 46.7 helical turns of duplexed RNA, 30 to 38 helical turns of duplexed RNA, 32 to 36 helical turns of duplexed RNA, and a combination thereof.

In another aspect, Rugged dsRNA is produced by isolating the 5-minute HPLC peak of a tdsRNA preparation.

### 3.3 Rugged DSRNA Preparation

In one embodiment, the starting material for making Rugged dsRNA may be dsRNA prepared in vitro using conditions of this disclosure. For example, the specifically configured dsRNA described in U.S. Pat. No. 4,024,222, 4,130,641, and 5,258,369 (which are incorporated by reference herein) are generally suitable as starting materials after selection for rugged dsRNA. tdsRNA (or preparations of tdsRNA) described in this disclosure is also useful as starting material.

After procuring starting material, Rugged dsRNA may be isolated by at least subjecting the partially hybridized strands of a population of dsRNA to conditions that denature most dsRNA (more than 10 wt % or mol %, more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 95 wt % or mol %, or more than 98 wt % or mol %) in the population, and then selection negatively or positively (or both) for dsRNA that remain partially hybridized. The denaturing conditions to unfold at least partially hybridized strands of dsRNA may comprise an appropriate choice of buffer salts, pH, solvent, temperature, or any combination thereof. Conditions may be empirically determined by observation of the unfolding or melting of the duplex strands of ribonucleic acid. The yield of rugged dsRNA may be improved by partial hydrolysis of longer strands of ribonucleic acid, then selection of (partially) hybridized stands of appropriate size and resistance to denaturation.

The purity of rugged dsRNA, which functions as tdsRNA, may thus be increased from less than about 0.1-10 mol % (e.g., rugged dsRNA is present in at least 0.1 mol % or 0.1 wt percent but less than about 10 mol % or 10 wt percent) relative to all RNA in the population after synthesis to a higher purity. A higher purity may be more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 98 wt % or mol %, or between 80 to 98 wt % or mol %. All wt % or mol % is relative to all RNA present in the same composition.

Another method of isolating Rugged dsRNA is to employ chromatography. Under analytical or preparative high-performance liquid chromatography, Rugged dsRNA can be isolated from a preparation (e.g., the starting material as described above) to produce poly(I):poly(C₁₂U)ₙ (e.g., poly(I):poly(C₁₁₋₁₄U)ₙ) as a substantially purified and pharmaceutically-active molecule with an HPLC peak of about 4.5 to 6.5 minutes, preferably between 4.5 and 6 minutes and most preferably 5 minutes.

Rugged dsRNA and the method of making rugged dsRNA are described in U.S. Pat. No. 8,722,874 and 9,315,538 (incorporated by reference).

### 3.4 Stabilizing Forms

Another embodiment is directed to a tdsRNA in a stable, recoverable, and optionally rapidly recoverable form. One stable form of tdsRNA is a tdsRNA composition with moisture content of less than about 1%, 3%, or 5%; or between 5-10%, 10-15%, 15-20%, or 20-25% by mass. This stable form may be made by a number of methods. One method is drying which can be performed, for example, by exposure to a dry environment or a dry stream of gas. Another method for drying may be performed by lyophilization which is also called vacuum drying. Yet another form of drying may be an alcohol precipitation. Alcohol precipitation may be performed, for example, by adjusting an RNA solution to 0.3 molar sodium acetate and then adding 3 volumes of alcohol to the RNA solution to yield a mixture of 25% RNA solution and 75% alcohol). This produces an alcohol precipitate of tdsRNA. The alcohol precipitate is incubated −20° C. overnight to improve the precipitation and increase yield. After chilling overnight, the mixture is centrifugation at 12,000g-14,000g for 10 min at 4° C. The pellet, containing the RNA can be dried and recovered by fluid hydration. The salts that can be used for precipitation may be sodium acetate at 0.3 molar, sodium chloride at 0.2 to 0.3 molar, ammonium acetate at up to 5 molar or lithium chloride at 0.10 molar. The alcohol may be, for example, ethanol or isopropanol. Another stable form of tdsRNA would be the alcohol precipitate of tdsRNA described above. tdsRNA can be retrieved from an alcohol precipitate of tdsRNA by converting the alcohol precipitate to dried tdsRNA as described above.

Stable tdsRNA can be retrieved or recovered from its stable form by fluid hydration. Hydration techniques include addition of a liquid to the stable tdsRNA. Fluid hydration allows stable tdsRNA to be rapidly utilized. Thus, in another embodiment, the invention provides a kit comprising a tdsRNA and a hydration fluid, wherein the tdsRNA is substantially free of moisture and is stable. The kit and the stable tdsRNA are stable for 1 year, 2 years, or 3 years at room temperature. When the fluid and the tdsRNA is combined, the rehydrated tdsRNA can be rapidly used. Thus, stabilized tdsRNA or kits comprising stabilized tdsRNA has substantial benefits over many types of pharmaceuticals and other RNA drugs which require storage at −20° C. or −80° C.

Exemplary elution/recovery liquids are aqueous. Nonlimiting examples of recovery fluids include water, a buffered solution, and phosphate buffered saline (PBS). One preferred recovery fluid is water. Naturally, pure water (H₂O) without any contaminants is preferred, and water with minimal contaminants that is medical grade is also preferred. The use of HPLC-grade or molecular biology-grade water certified to be RNase free as a diluent, or as the liquid part of PBS or TE buffer is also preferable. Another preferred recovery fluid is PBS which can be 0.15 M NaCl, 0.01 M Na₃PO₃, 0.001 M MgCl₂; or can be 0.138 M NaCl; 0.0027 M KCl, pH 7.4.

The reconstituting or recovery step may include the steps of contacting a diluent (e.g., PBS or water) with the dried tdsRNA; and, optionally, agitating the vial containing the dried tdsRNA to dissolve the lyophilized medicament. Contacting a diluent to dried tdsRNA may involve, for example, injecting a diluent into a vial containing lyophilized tdsRNA.

It is preferred that in any embodiment, the diluent, including water or PBS, is nuclease free and endotoxin free. Testing for nuclease free water may be by commercially available products such as RNaseAlert® and DNaseAlert™ reagents. Screened for endotoxins may be performed with a Limulus amebocyte lysate (LAL) assay.

In another embodiment, tdsRNA may be stored as an alcohol (e.g., ethanol) precipitate at −80° C. For example, an alcohol precipitate can be made by adding 0.1 volume of 3 M sodium acetate solution to an RNA solution, mixing, and adding 2.5 to 3 volumes of ethanol and mixing. Then the solution with the precipitate may be chilled to −20° C. or −80° C. for storage.

Other formulations of recovery fluids may comprise the following components buffers, chelating agents, reducing agents, or nuclease inhibitors. While these agents are described for recovery fluids, any of these agents may be combined with tdsRNA in the compositions of this disclosure.

Buffers can maintain pH within a particular range, for example, between 1 and 12, and are also referred to as pH stabilizing agents. More typically, pH will range within about pH 5.0 to about pH 12.0. A particular example of a pH stabilizing agent is a zwitterion. Specific nonlimiting examples of pH stabilizing agents include Tris (hydroxymethyl) aminomethane hydrochloride (TRIS), N-(2-hydroxyethyl) piperazine-N′-2-ethanesulfonic acid (HEPES), 3-(N-morpholino) propanesulfonic acid (MOPS), 2-(N-morpholino) ethanesulfonic acid (MES), N-tris[hydroxymethyl]methyl-2-aminoethanesulfonic acid (TES), N-[carboxymethyl]-2-aminoethanesulfonic acid (ACES), N-[2-acetamido]-2-iminodiacetic acid (ADA), N,N-bis[2-hydroxyethyl]-2-aminoethanesulfonic acid (BES), N-[2-hydroxyethyl]piperazine-N′-[2-hydroxypropoanesulfonic acid] (HEPPSO), N-tris[hydroxymethyl]methylglycine (TRICINE), N,N-bis[2-hydroxyethyl]glycine (BICINE), 4-(cyclohexylamino)-1-butanesulfonic acid (CABS), 3-(cyclohexylamino)-1-propanesulfonic acid (CAPS), 3-(cyclohexylamino-2-hydroxy-1-propanesulfonic acid (CAPSO), 2-(cyclohexylamino) ethanesulfonic acid (CHES), N-(2-hydroxyethyl)piperazine-N′-(3-propanesulfonic acid) (EPPS), piperazine-N,N′-bis (2-ethanesulfonic acid (PIPES), [(2-hydroxy-1,1-bis [hydroxymethyl]ethyl) amino]-1-propanesulfonic acid (TAPS), N-tris (hydroxymethyl) methyl-4-aminobutane sulfonic acid (TABS), 2-amino-2-methyl-1-propanol (AMP), 3-[(1,1-dimethyl-2-hydroxyethyl)amino]-2-hydroxypropanesulfonic acid (AMPSO), ethanolamine and 3-amino-1-propanesulfonic acid. Additional specific nonlimiting examples of pH stabilizing agents include potassium chloride, citric acid, potassium hydrogenphthalate, boric acid, potassium dihydrogenphosphate, Diethanolamine, sodium citrate, sodium dihydrogenphosphate, sodium acetate, sodium carbonate, sodium tetraborate, cacodylic acid, imidazole and 2-Amino-2-methyl-1-propanediol.

Buffers or pH stabilizing agents are typically used in a range of about 0.1 mM to about 500 mM, in a range of about 0.5 mM to about 100 mM, in a range of about 0.5 mM to about 50 mM, in a range of about 1 mM to about 25 mM, or in a range of about 1 mM to about 10 mM. More particularly, buffers can have a concentration of about (i.e., within 10% of) 1 mM, 2 mM, 5 mM, 10 mM, 15 mM, 20 mM, 25 mM, 30 mM, 40 mM, or 50 mM. For elution or recovery of tdsRNA, such ranges and buffer concentrations for elution and recovery liquids are appropriate.

Chelating agents may be used in hydration fluids because they inhibit nucleases and other undesirable activities. Chelating agents typically form multiple bonds with metal ions, and are multidentate ligands that can sequester metals. Metal sequestration can in turn reduce or prevent microbial growth or degradation of tdsRNA. Specific nonlimiting examples of chelating agents include EDTA (Ethylenediamine-tetraacetic acid), EGTA (Ethyleneglycol-O,O′-bis (2-aminoethyl)-N,N,N′,N′-tetraacetic acid), GEDTA (Glycoletherdiaminetetraacetic acid), HEDTA (N-(2-Hydroxyethyl) ethylenediamine-N,N′,N′-triacetic acid), NTA (Nitrilotriacetic acid), Salicylic acid, Triethanolamine and porphines. Typical concentrations of chelating agents are in a range of about 0.1 mM to about 100 mM, in a range of about 0.5 mM to about 50 mM, or in a range of about 1 mM to about 10 mM.

Reducing agents and antioxidants typically inhibit microbial growth and reduce biomolecule (e.g., tdsRNA) oxidation. Particular nonlimiting classes of such agents include free radical scavenging agents. Specific nonlimiting examples of reducing agents and anti-oxidants include DTT (dithiothreitol), dithioerythritol, urea, uric acid, 2-mercaptoethanol, dysteine, vitamin E, vitamin C, dithionite, thioglycolic acid and pyrosulfite.

Nuclease inhibitors inhibit degradation of nucleic acid. Particular nonlimiting classes of nuclease inhibitors include ribonuclease inhibitor (e.g., RNaseOUT, Invitrogen Catalog #10777-019; RNase Block, Stratagene Catalog #300151), diethyl pyrocarbonate and aurintricarboxylic acid (ATA).

As used herein, the term “substantially free,” and grammatical variations thereof, when used in reference to moisture content of a tdsRNA, means that the tdsRNA has less than about 25% moisture content (i.e., 23-27%) by mass, relative to the total mass of the tdsRNA. Typically, moisture (water) content will be less than 25%, for example, less than 20-25%, 15-20%, 10-15%, 5-10%, 2-5%, 1-2%, or less than 1% moisture.

The invention also provides methods of storing tdsRNA a recoverable form. In one embodiment, a method includes providing a recoverable form of tdsRNA in an ethanol precipitate. In another embodiment, the disclosure provides a recoverable form of tdsRNA in a dried (low moisture content) form, optionally reducing moisture to less than 1%, less than 3%, less than 5%, 5-10%, 10-15%, 15-20%, or 20-25% by mass, thereby producing a stored tdsRNA in a recoverable form. The invention additionally provides methods for recovering a tdsRNA. In one embodiment, a method includes providing a stabilized and dried tdsRNA, hydrating the dried tdsRNA or at least a portion of the of the dried tdsRNA with a hydration fluid, and using the hydrated tdsRNA for administrating to subjects.

Stabilized tdsRNA, especially the dried stabilized tdsRNA (e.g., lyophilized) which can be stored at room temperature has significant benefits at least because many doses of tdsRNA can be stored in a small, lightweight, and compact form. For example, the stabilized tdsRNA can be shipped anywhere in the world in a small package in response to a virus outbreak. Also, the stabilized tdsRNA, because of their stability, may be stored in warehouses for immediate deployment when needed. The tdsRNA can be administered to people exposed to the virus. As a precaution, since many doses can be transported at less expense compared to more fragile medicaments, tdsRNA may be administered to as many people as desired regardless of their virus exposure as a precaution. In these situations, if possible, recovery fluid or kids comprising tdsRNA and a recovery fluid can be shipped with the tdsRNA or maybe even packaged together in a kit. One kit example would be a vial with a dry portion and a fluid portion and where the two portions can be mixed without exposing the contents to outside contaminants. Another choice would be for the recovery fluid to be locally sourced which would cause savings in shipping and handling.

### 3.5 Stabilizing Polymers

In any of the described embodiments, the tdsRNA may be complexed with a stabilizing polymer such as: polylysine, polylysine plus carboxymethylcellulose (lysine carboxy methyl cellulose), polyarginine, polyarginine plus carboxymethylcellulose, or a combination thereof. Some of these stabilizing polymers are described, for example, in U.S. Pat. No. 7,439,349.

### 3.6 Modified Backbone

The tdsRNA may comprise one or more alterations in the backbone of the nucleic acid. For example, configured tdsRNA may be made by modifying the ribosyl backbone of poly(riboinosinic acid) r(Iₙ), for example, by including 2′-O-methylribosyl residues. Specifically configured dsRNA may also be modified at the molecule's ends to add a hinge(s) to prevent slippage of the base pairs, thereby conferring specific bioactivity in solvents or aqueous environments that exist in human biological fluids.

### 4. Additional Agents

The tdsRNA of this disclosure may be in a compound or in combination with a number of additional agents. Some of the preferred agents are described herein.

### 4.1 Carrier or Vehicle

Suitable agents may include a suitable carrier or vehicle for intranasal mucosal delivery. As used herein, the term “carrier” refers to a pharmaceutically acceptable solid or liquid filler, diluent or encapsulating material. In one aspect, the carrier is a suitable carrier or vehicle for intranasal mucosal delivery.

### 4.2 Absorption-Promoting Agents

Suitable agents may include any suitable absorption-promoting agents. The suitable absorption-promoting agents may be selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacyl methyl sulfoxide, azone (1-dodecylazacycloheptan-2-one or laurocapram), sodium lauryl sulfate, oleic acid, and the bile salts, may be employed to enhance mucosal penetration of the tdsRNA. In additional aspects, surfactants (e.g., polysorbates) are employed as adjunct compounds, processing agents, or formulation additives to enhance intranasal delivery of the tdsRNA.

### 4.3 Delivery-Enhancing Agents

As used herein, the term “delivery-enhancing agents” refers to any agents which enhance the release or solubility (e.g., from a formulation delivery vehicle), diffusion rate, penetration capacity and timing, uptake, residence time, stability, effective half-life, peak or sustained concentration levels, clearance and other desired intranasal delivery characteristics (e.g., as measured at the site of delivery, or at a selected target site of activity such as the bloodstream) of tdsRNA or other biologically active compound(s).

### 4.4 Mucolytic or Mucus Clearing Agents

In another embodiment, the present compositions may also comprise other suitable agents such as mucolytic and mucus-clearing agents. The term “mucolytic and mucus-clearing agents,” as used herein, refers to any agents which may serve to degrade, thin or clear mucus from intranasal mucosal surfaces to facilitate absorption of intranasally administered biotherapeutic agents including tdsRNA. Based on their mechanisms of action, mucolytic and mucus clearing agents can often be classified into the following groups: proteases (e.g., pronase, papain) that cleave the protein core of mucin glycoproteins, sulfhydryl compounds that split mucoprotein disulfide linkages, and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

### 4.5 Ciliostatic Agents

In another embodiment, the present compositions may comprise ciliostatic agents.

As used herein, the term “ciliostatic agents” refers to any agents which are capable of moving a layer of mucus along the mucosa to removing inhaled particles and microorganisms. Examples of ciliostatic factors include a phenazine derivative, a pyo compound (2-alkyl-4-hydroxyquinolines), and a rhamnolipid (also known as a hemolysin).

### 4.6 Penetration or Permeation-Promoting Agent

In another embodiment, the intranasal mucosal therapeutic and prophylactic formulations of the present disclosure may be supplemented with any suitable penetration-promoting agent that facilitates absorption, diffusion, or penetration of tdsRNA across mucosal barriers. Examples of such agents include sodium salicylate and salicylic acid derivatives (acetyl salicylate, choline salicylate, salicylamide, etc.), amino acids and salts thereof (e.g., monoaminocarboxlic acids such as glycine, alanine, phenylalanine, proline, hydroxyproline, etc., hydroxyamino acids such as serine, acidic amino acids such as aspartic acid, glutamic acid, etc., and basic amino acids such as lysine, etc.—inclusive of their alkali metal or alkaline earth metal salts), and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts).

### 4.7 Vasodilator Agents

In another embodiment, the present formulation may also comprise other suitable agents such as vasodilator agents. Examples of vasodilators include calcium antagonists, potassium channel openers, ACE inhibitors, angiotensin-II receptor antagonists, alpha-adrenergic and imidazole receptor antagonists, beta-1-adrenergic agonists, phosphodiesterase inhibitors, eicosanoids and NO donors.

### 4.8 RNASE Inhibitory Agent and Enzyme Inhibitor

The compositions of the present disclosure may contain an RNase inhibitor or an enzyme inhibitor. Typical enzyme inhibitors that are commonly employed and that may be incorporated into the present disclosure may be, for example, leupeptin, aprotinin, and the like. RNase inhibitors are commonly used as a precautionary measure in enzymatic manipulations of RNA to inhibit and control RNase. These are commercially available from a number of sources such as, for example, Invitrogen (SUPERase, In RNase Inhibitor, RNaseOUT, RNAsecure, and RNase Inhibitor).

### 5. Administration (Delivery)

Administration to the subject or administering to the subject may include one or more of the following: intranasal administration (pulmonary airway administration); intranasal administration and oral administration; oral administration (through the mouth, by breathing through the mouth); topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; and dry powder inhalants administration. The most preferred method is intranasal administration. Intranasal administration in this disclosure refers to administering to nasal passages or administering to nasal epithelium. As another example, administering may be performed by a delivery system or medical device comprising the tdsRNA. The delivery system or medical device may be a nebulizer; a sprayer; a nasal pump; a squeeze bottle; a nasal spray; a syringe sprayer or plunger sprayer (a syringe providing pressure to an attached sprayer or nozzle), a swab; a pipette; a nasal irrigation device; a nasal rinse; or any device for administrating a composition to the inside of the nose.

In contrast, other emulsifying agents typically protect the emulsified droplets by forming a liquid crystalline layer around the emulsified droplets. In compositions that employ a liquid crystalline layer-forming emulsifying agent, the hydrophilic-lipophilic balance (HLB) of the oil phase of the emulsion must be matched with that of the emulsifying agent to form a stable emulsion and, often, one or more additional emulsifying agents (secondary emulsifying agents) may be added to further stabilize the emulsion.

The liquid compositions are particularly suited for nasal administration.

### 5.1 Administration Delivery System

Administration may also be from any known delivery system. A delivery system may be at least one selected from the group consisting of: a pill, a capsule, a needle, a cannula, an implantable drug depot, an infusion system (e.g., a device similar to an insulin pump); a nebulizer; a sprayer; a nasal pump; a squeeze bottle; a nasal spray; a syringe sprayer, a plunger sprayer (a syringe providing pressure to an attached sprayer or nozzle); a nasal aerosol device; a controlled particle dispersion device; a nasal aerosol device; a nasal nebulization device; a pressure-driven jet nebulizer; an ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered-dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and a combination thereof.

### 5.2 Formulations and Dosage

Formulations for administration (i.e., pharmaceutical compositions) may include a pharmaceutically acceptable carrier with the tdsRNA.

Pharmaceutical carriers include suitable non-toxic vehicles in which a composition of the disclosure is dissolved, dispersed, impregnated, or suspended, such as water or other solvents, fatty materials, celluloses and their derivatives, proteins and their derivatives, collagens, gelatine, polymers, adhesives, sponges, fabrics, and the like and excipients which are added to provide better solubility or dispersion of the drug in the vehicle. Such excipients may include non-toxic surfactants, solubilizers, emulsifiers, chelating agents, binding materials, lubricants, softening agents, and the like. Pharmaceutically acceptable carriers may be, for example, aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents.

The tdsRNA may be a combination or any subset of dsRNA described above (e.g., formula (1) to formula (5)). It is understood that in one aspect, tdsRNA may comprise a combination of all of the examples of tdsRNA described above or any subset of the above examples. With respect to the subsets, the specific exclusion of one or more specific embodiments of tdsRNA is also envisioned. As nonlimiting examples, tdsRNA may comprise any of the following: (A) all of the examples of tdsRNA as described above, (B) all of the examples of tdsRNA described above but without rIₙ·r(C₁₁₋₁₄U)ₙ, (C) Rugged dsRNA, (D) rIₙ·r(C₁₂U)ₙ, (E) tdsRNA as described above but without rIₙ·r(C₁₁₋₁₄U)ₙ and without Rugged dsRNA, (F) rIₙ·r(C₁₂U)ₙ, and Rugged dsRNA; or (G) rIₙ·r(C₁₁₋₁₄U)ₙ and Rugged dsRNA.

### 5.3 Medicament

In another aspect, a medicament (e.g., a pharmaceutical composition) containing the tdsRNA is provided. Optional other components of the medicament include excipients and a vehicle (e.g., aqueous buffer or water for injection) packaged aseptically in one or more separate containers (e.g., nasal applicator). Further aspects will be apparent from the disclosure and claims herein.

### 5.4 Dosage for the Average Subject

The dosages are generally applicable to a subject as described in another section of this disclosure. In a preferred embodiment, the subject is human.

For a subject (especially human) the preferred intranasal dosage of tdsRNA may be: 0.1 μg to 1,200 μg; 0.1 to 25 μg; 25 μg to 50 μg; 50 μg to 100 μg; 100 μg to 200 μg; 200 μg to 400 μg; 400 μg to 800 μg; 800 μg to 1,250 μg. For example, intranasal dosages may be 25 μg; 50 μg; 125 μg; 200 μg; 250 μg; 500 μg; 1,000 μg; 1,250 μg; 1500 μg; 2000 μg; 2500 μg; or 3000 μg. Nasal dosages may be administered in any combination between the two nostrils. For example, 0% to 100% in one nostril and the remainder in the second nostril. In a preferred embodiment Each nostril receiving 50% of the dosage is one preferred embodiment.

tdsRNA may be administered by any method. One preferred method is iv administration. The dosage per administration by any method including iv administration may be in the following range per administration: 0.1 μg to 1,200 mg; 0.1 to 25 mg; 25 mg to 50 mg; 50 mg to 100 mg; 100 mg to 200 mg; 200 mg to 400 mg; 400 mg to 800 mg; 800 mg to 1,200 mg. For example, iv dosages may be 25 mg; 50 mg; 125 mg; 200 mg; 250 mg; 400 mg; 500 mg; 1,000 mg; 1,200 mg.

The amount of tdsRNA per administration may be at least one selected from 0.1 mg/kg, 0.2 mg/kg, 0.4 mg/kg, 0.6 mg/kg, 0.8 mg/kg, 1 mg/kg, 2 mg/kg, 3 mg/kg, 4 mg/kg, 5 mg/kg, 6 mg/kg, 8 mg/kg, 10 mg/kg.

In one embodiment, the tdsRNA is administered iv at a dose from about 1 mg/kg to 10 mg/kg biweekly. As another example, the administration may be in 50-1400 milligrams every other day, leading to an average daily dosage of 25-700 milligrams per day. In one embodiment, the tdsRNA is administered at a dose from about 0.50 mg/kg to 10 mg/kg every other week. 50-1400 milligrams every other day, leading to an average daily dosage of 25-700 milligrams per day.

The tdsRNA is administered at a frequency selected from the group consisting of: one dose per day, one dose every 2 days, one dose every 3 days, one dose every 4 days, one dose every 5 days, 4 doses a week, 3 doses a week, 2 doses a week, 1 dose a week, one dose every two weeks, one dose every three weeks, one dose every four weeks, and one dose every month.

One preferred second treatment is iv tdsRNA, 200 mg per dose, two times a week for two weeks. This is followed by 400 mg per dose, two times a week for another 2, 4, 8, or 12 weeks or for as long as necessary. Alternatively, the iv tdsRNA may be administered 400 mg per dose, two times a week for another 2, 4, 8, or 12 weeks or for as long as necessary.

### 5.5 Amount Per Unit Dose

The amount per unit dose of tdsRNA may be at least one selected from 0.1 mg/kg, 0.2 mg/kg, 0.4 mg/kg, 0.6 mg/kg, 0.8 mg/kg, 1 mg/kg, 2 mg/kg, 3 mg/kg, 4 mg/kg, 5 mg/kg, 6 mg/kg, 8 mg/kg, 10 mg/kg.

### 5.6 Dose Frequency

In certain embodiments, the tdsRNA is administered at a frequency selected from the group consisting of: one dose per day, one dose every 2 days, one dose every 3 days, one dose every 4 days, one dose every 5 days, 4 doses a week, 3 doses a week, 2 doses a week, 1 dose a week, one dose every two weeks, one dose every three weeks, one dose every four weeks, and one dose every month. Nasal administration may be as listed above or may be 2 doses per day or three doses per day. Administration or dosing can be continued as long as they have a beneficial effect on the subject.

### 6. Nasal Administration Devices

A device or delivery system, encompassing a composition of the disclosure is also an embodiment.

The composition of the disclosure may be delivered by any nasal administration device or combination of devices. A combination refers to a composition that is both administered by two different devices or a device having the feature of two devices. Nonlimiting examples of suitable devices that can be use individually or together include at least one selected from the group consisting of: a nebulizer; a sprayer (e.g., a spray bottle such as “Nasal Spray Pump w/Safety Clip, Pfeiffer SAP #60548; a squeeze bottle (e.g., bottle commonly used for nasal sprays, including ASTELIN (azelastine hydrochloride, Medpointe Healthcare Inc.) and PATANASE (olopatadine hydrochloride, Alcon, Inc.); a nasal pump spray (e.g., APTAR PHARMA nasal spray pump); a controlled particle dispersion devices (e.g., VIANASE electronic atomizer); a nasal aerosol device (e.g., ZETONNA nasal acrosol); a nasal nebulization device (e.g., EASYNOSE nebulizer, a pressure-driven jet nebulizer, or an ultrasonic nebulizer); a powder nasal delivery devices (e.g., OPTINOSE breath-powered nasal delivery device); an atomized nasal medication device (e.g., LMA MAD NASAL device); an instillation device; an inhalation device (e.g., an inhaler); a powder dispenser; a dry powder generator; an aerolizer (e.g., intrapulmonary aerosolizer or a sub-miniature aerosolizer, metered aerosol, powdered aerosol, spray acrosol); a spray; a metered spray; a metered dose inhalers (e.g., a propellant based metered-dose inhaler); a dry powder inhalation device; an intranasal instillation device; an intravesical instillation device; an insufflation device.

An application device for application to mucous membranes, such as, that of the nose, throat, and/or bronchial tubes (i.e., inhalation). This can be a swab, a pipette or a device for nasal irrigation, nasal rinse, or nasal lavage.

Another example is a syringe or plunger-activated sprayer. This could be, for example, a sprayer head (or nozzle) attached, for example, via a Luer lock, to a syringe. The syringe applies pressure to a composition that flows through the sprayer head and produces a spray or an acrosol.

### 7. Discussion of Further Embodiments and Features

### 7.1 Subject or Patient

As used herein, a “subject” has the same meaning as a “patient” and is a mammal, preferably a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Other examples of subjects include any animal such as civet cats, swine, cattle, horses, camels, cats, dogs, rodents, birds, bats, rabbits, ferrets, mink, snake, and the like. As used herein, the terms “patient” or “subject” are used interchangeably.

### 7.2 Devices and Kits

In another aspect, the present disclosure relates to and comprises a therapeutic device for intranasal delivery. In one embodiment, the therapeutic device may comprise any suitable devices charged with a preparation of tdsRNA and optionally, another biologically active agent such as a vaccine or antigen. These devices are described in more detail below.

### 7.3 Effective Amount: Therapeutically or Prophylactically Effective Amount

The compositions are delivered in effective amounts. The term “effective amount” refers to the amount necessary or sufficient to realize a desired biological effect which is, for example, developing a broad-based response, or reducing, stopping the advance of, or reversing the symptoms of a viral infection. In addition to the sample dosages and administration methods mentions, one of ordinary skill in the art can empirically determine the effective amount of the tdsRNA without necessitating undue experimentation. It is preferred that a maximum dose be used, that is, the highest safe dose according to medical judgment.

Effective dosage forms, modes of administration, and dosage amounts may be determined empirically, and making such determinations is within the skill of the art. It is understood by those skilled in the art that the dosage amount will vary with the route and mode of administration, the rate of excretion, the duration of the treatment, the identity of any other drugs (e.g., antiviral agent) being co-administered, the age, size, species of mammal (e.g., human patient), and other factors well known in the arts of medicine and veterinary medicine. In general, a suitable dose of any active agent disclosed herein or a composition containing the same will be that amount of the active agent (tdsRNA) or composition comprising the active agent, which is the lowest dose effective to produce the desired effect. The desired effect may be to reduce the severity or duration of a symptom of a viral infection or developing a broad-based immune response (broad-based immunity) against a virus variant.

### 8. Other Aspects

In this specification, stating a numerical range, it should be understood that all values within the range are also described (e.g., one to ten also includes every value between one and ten as well as all intermediate ranges such as two to ten, one to five, and three to eight). The term “about” may refer to the statistical uncertainty associated with a measurement or the variability in a numerical quantity that a person skilled in the art would understand does not affect the operation of the disclosure or its patentability.

All modifications and substitutions that come within the meaning of the claims and the range of their legal equivalents are to be embraced within their scope. A claim which recites “comprising” allows the inclusion of other elements to be within the scope of the claim. The disclosure is also described by such claims reciting the transitional phrases “consisting essentially of” (i.e., allowing the inclusion of other elements to be within the scope of the claim if they do not materially affect the operation of the disclosure) or “consisting of” (i.e., allowing only the elements listed in the claim other than impurities or inconsequential activities which are ordinarily associated with the disclosure) instead of the “comprising” term. Any of these three transitions can be used to claim the disclosure.

An element described in this specification should not be construed as a limitation of the claimed disclosure unless it is explicitly recited in the claims. Thus, the granted claims are the basis for determining the scope of legal protection instead of a limitation from the specification which is read into the claims. In contradistinction, the prior art is explicitly excluded from the disclosure to the extent of specific embodiments that would anticipate the claimed disclosure or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements, embodiments, and aspects disclosed herein are also considered to be aspects and embodiments of the disclosure. Similarly, generalizations of the disclosure's description are considered to be part of the disclosure.

From the foregoing, it would be apparent to a person of skill in this art that the disclosure can be embodied in other specific forms without departing from its spirit or essential characteristics.

While the disclosure has been described in connection with what is presently considered to be the most practical and preferred embodiment, it is to be understood that the disclosure is not to be limited to the disclosed embodiment, but on the contrary, is intended to cover various modifications and equivalent arrangements included within the spirit and scope of the appended claims.

### 9. Incorporation By Reference

All publications, patent applications, and patents mentioned herein are hereby incorporated by reference in their entirety as if each individual publication or patent was specifically and individually indicated to be incorporated by reference. These patents include, at least, U.S. Pat. Nos. 4,024,222, 4,130,641, 5,258,369, 7,439,349, 8,722,874 and 9,315,538. In case of conflict, the present application, including any definitions herein, will control.

### EXAMPLES

### Example 1 Nasal Administration of tdsRNA

Ampligen® is a well-defined selective Toll-like receptor 3 (TLR3) agonist inducing innate immune antiviral responses. Ampligen has been administered intravenously in approximately 100,000 doses in clinical trials and compassionate use programs. Ampligen has been shown to be generally well tolerated intranasally in humans. Besides, intranasal administration of Ampligen as a flu vaccine adjuvant was found to be well tolerated.

TLR3 is expressed at a high level in human airway epithelial cells, including the nose and nasal pharynx. TLR3 serves as a pathogen recognition receptor to stimulate the innate immune response against many respiratory pathogens, including coronaviruses. As a highly specific TLR3 agonist, Ampligen stimulates the production of type I interferons, which exert both antiviral and immunomodulatory activity.

A phase I trial was performed to assess the safety, tolerability and biological activity of repeated administration of Ampligen intranasally every other day for 13 days (7 doses) in healthy volunteers. Also, this study was performed for the further development of Ampligen as a potential treatment modality for COVID-19 and other pulmonary viral diseases.

Inclusion criteria were: 1. Signed informed consent prior to any study-mandated procedure. 2. Male or female subjects, 18 to 70 years of age, inclusive at screening. 3. Body mass index (BMI) between 18 and 32 kg/m2, inclusive at screening, and with a minimum weight of 50 kg. 4. Participant must be healthy, in the investigator's clinical judgment, as confirmed by medical history, physical examination, vital signs, ECG and laboratory assessments performed at screening. 5. Willing to comply with effective contraception during the study if subject is male or women of childbearing potential, up to 90 days after the last dose of study treatment. 6. Has the ability to communicate well with the investigator and is willing to comply with the study restrictions.

This experiment was performed to determine the safety and possible efficacy of intranasal administration of tdsRNA and the results show that tdsRNA is generally well tolerated.

A total of 40 human patients were enrolled. This study showed that AMPLIGEN® is well tolerated when administered intranasally at dose levels up to 1250 μg every other day for seven doses over 13 days.

### Example 2 A Stable Form of tdsRNA

To determine if tdsRNA is stable at room temperature after drying, we analyzed the stability of AMPLIGEN® (rintatolimod) in PBS. Briefly, AMPLIGEN® in PBS (0.15 M NaCl, 0.01 M Na₃PO₃, 0.001 M MgCl₂) was lyophilized and stored at room temperature for 3 months. After storage, the AMPLIGEN® was reconstituted with water and examined for activity and structure. No differences in activity or structure were observed.

The results above also indicate that tdsRNA can be stored long-term in a preserved form that resists degradation. The tdsRNA is therefore suitable for application requiring high-quality non-degraded tdsRNA. Furthermore, the recovered tdsRNA could directly be used for treatment of subjects.

### Example 3 tdsRNA Provides Protection Against Nasal Infections

The route of human infection of respiratory viruses is believed to be primarily by entry into the nasal epithelium. By dosing tdsRNA (Ampligen® (rintatolimod) in this example) every other day intranasally, we are studying whether respiratory viruses can be inhibited at the point of entry, and thus will be much less likely to progress to a pulmonary infection or moderate respiratory disease. These characteristics make Ampligen a potent candidate to be developed for an early treatment strategy and (post-exposure) prophylaxis against many respiratory viruses. Because Ampligen does not act by binding to proteins or specific nucleic acid sequences of viruses it can also be developed for potential future outbreaks of other respiratory viruses including, for example, future variants and strains of rhinovirus, SARS-COV-2, or other coronaviruses.

A study was performed to determine the effects of Ampligen upon intranasal administration. Specifically, the effect of most interest is to see if Ampligen can protect subjects from nasal infections. Since this study has been designed to evaluate the effectiveness of tdsRNA (e.g., Ampligen® (rintatolimod)) the compound for future (post-exposure) prophylaxis, it was decided to define a heterogenous study population including men and women, of a relatively broad age range (18-70 years). An age limit of 70 has been chosen to account for a potential understated mucosal immune response in elderly participants due to immunosenescence. In addition, participants will be excluded if there are pre-existing immunological or anatomical conditions that may interfere with the study outcomes or participant safety.

All cohorts and subject selection was randomized, double-blind and placebo-controlled. Randomization is deemed appropriate to avoid selection bias for active compound or placebo treatment. By double-blinding the study, bias arising from the study subject's or investigator's knowledge about treatment assignment is avoided. The investigator, sponsor team, all site staff, and everyone else with direct involvement in study conduct was fully blinded throughout the study, with the exception of the study pharmacist and statistician. A matching dosage form, indistinguishable from active treatment, was used as a placebo treatment.

A total of 40 subjects participated in this study in 4 cohorts. Each cohort has ten (10) subjects. Before treatment, subjects attended a single baseline visit in the period of day −4 till day −1 before treatment initiation. This visit will include screening for the presence of respiratory pathogens in nasal swabs. Only subjects that are negative for respiratory pathogens are allowed to proceed.

During the treatment period subjects regularly visit the clinical site for study treatment administrations (every other day), safety checks and sampling for safety and mucosal immune measurements. After every treatment administration, subjects was kept admitted to the clinical unit for a period of minimally 6 hours on the first day of administration (day 1) and one-hour post-dose for subsequent treatment administrations. The total treatment duration is 13 days (i.e., 7 administrations), the observation period 16 days.

The first cohort (cohort 1, 75 μg) started with a sentinel procedure. One subject received Ampligen and another subject received placebo. Repeated intranasal administration was evaluated after three consecutive doses. Since there were no safety issues and the administration was well tolerated, cohort 1 was expanded with an additional 8 subjects (7 receiving Ampligen, 1 placebo) leading to a total of 10 subjects (8 receiving Ampligen, 2 placebo). Another evaluation of the sentinel group was performed after all 7 consecutive doses have been administered. Once again, since there were no adverse effects, continuing dosing of the remaining 8 subjects of cohort 1 was commenced. Safety and tolerability endpoints of the total cohort was assessed up to day 15. Since there were no adverse events, the study of the next cohort with a higher Ampligen dose was started. The subjects visited the study sites for follow-up visits on day 28 (±3 days).

The second, third, and fourth cohorts underwent the same procedure except dosage was at 200 μg, 500 μg and 1250 μg respectively. In each cohort of 10 subjects, 8 were administered the tdsRNA while 2 will receive placebo (saline). No adverse events occurred during this experiment.

Ampligen or a placebo was administered to the subjects intranasally via a nasal sprayer, at the clinical unit. A volume of 250 μL with half the tdsRNA dosage was administered in each nostril for a total administration of 500 μL per dose. The dosage-the amount of Ampligen in total for each administration—is as described above. Each individual will receive the same dose throughout the treatment period. Subjects received a total of 7 doses of Ampligen or placebo. Dosing was started on day 1 and was administered every other day until day 13. A placebo (normal saline) was used as a negative control in this study. The placebo was indistinguishable from the active compound.

Without wishing to be bound by theory, we postulate that this vaccination strategy induced, among other effects, a cross-reaction secretory IgA against highly pathogenic respiratory viruses. The rationale for dosing Ampligen intranasally every other day is based on establishing an antiviral state in the nose and nasopharynx that will inhibit the replication of respiratory viruses for greater than or equal to two days (i.e., until the next dose). We believe that respiratory viruses can be inhibited at the point of entry, and thus was much less likely to progress to a pulmonary infection, or moderate/severe COVID-19 disease.

Of the 32 subjects administered Ampligen, only one subject suffered from a respiratory infection which was identified as a rhinovirus-an infection rate of about 3.12%. In contrast, among the placebo group, 12.5% suffered from a respiratory infection which was identified as a corona NL63 virus. This represents a clinically significant 75% reduction in viral infections among the subjects administered Ampligen instead of placebo. The route of human infection of respiratory viruses is primarily nasal. By dosing Ampligen intranasally, the respiratory viruses were inhibited at the point of entry and will thus be much less likely to result in pulmonary infection.

1. (canceled)

2. A method of treating a nasal virus infection in a subject, comprising administering to said subject a tdsRNA, wherein administering is at least two nasal administrations to nasal mucosa of the subject during viral replication in the nasal mucosa of the subject
wherein the tdsRNA is at least one selected from the group consisting of
rIₙ·r(CₓU)ₙ (formula 1);
rIₙ·r(CₓG)ₙ (formula 2);
rAₙ·rUₙ (formula 3);
rIₙ·rCₙ (formula 4);
and
rugged dsRNA (formula 5);
wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

3. The method of claim 2, wherein administering is at least 2, 3, 4, 5, 6, 7, 8, 9 or 10 administrations of tdsRNA.

4. The method of claim 2, further comprising a step of determining there is a nasal virus or active nasal virus replication in the nasal passages of the subject before, during, or after the administering step.

5. The method of claim 2, wherein administering is continued until nasal virus is reduced by 90%.

6. The method of claim 2, wherein said method reduces a symptom or a sign of the virus infection in the subject, in a part of the subject, or in the respiratory tract of the subject.

7. The method of claim 6, wherein the symptom or the sign is at least one selected from the group consisting of:
virus protein level; virus nucleic acid level; peak viral load; time to peak viral load; duration of viral shedding; viral load area under the curve (viral AUC); virus titer; nasal virus protein level; nasal virus nucleic acid level; nasal peak viral load; nasal time to peak viral load; nasal duration of viral shedding; nasal viral load area under the curve (viral AUC); nasal virus titer; cough; runny nose; nasal congestion; sore throat; headache; body aches and pains; fever; chills; fatigue; rhinorrhea; cough; and malaise.

8. The method of claim 12, wherein the administering is
started within 1 day, 2 days, or 3 days after exposure to the virus;
started within 1 day, 2 days, 3 days after onset of a symptom of virus infection;
or
started within 1 day, 2 days, or 3 days after a positive test for the virus.

9. The method of claim 12,
wherein the method induces a protective immune response in the subject against the virus or a second virus.

10. The method of claim 9, wherein the protective immune response is an enhanced innate immune response, an enhanced adaptive immune response, or an enhanced mucosal immune response.

11. The method of claim 2, wherein the method induces, in the subject, at least one selected from the group consisting of: enhanced cross protection; enhanced epitope spreading;
enhanced cross reactivity; and enhanced mucosal immunity.

12. The method of claim 2, wherein the method produces a broad-based immune response in the subject.

13. The method of claim 2, wherein the broad-based immune response is an immune response in the subject to a second virus, wherein the second virus is a variant of the virus or a different virus.

14. The method of claim 2, wherein the method causes a reduction in a symptom or a sign of a second virus infection.

15. The method of claim 2, where the symptom or the sign of the second virus infection is at least one selected from the group consisting of:
second virus protein level; second virus nucleic acid level; peak second viral load; time to peak second viral load; duration of second viral shedding; second viral load area under the curve (viral AUC); second virus titer; cough; runny nose; nasal congestion; sore throat; headache; body aches; body pains; fever; chills; fatigue; rhinorrhea; cough; and malaise.

16. The method of claim 2, claims, wherein the subject is a mammal, preferably a human.

17. The method of claim 2, wherein the virus or the second virus is at least one selected from the group consisting of:
adenovirus; coronavirus; Ebola Virus; H10N8 influenza; H1N1 influenza; H3N2 influenza; H5 influenza; H5N1 influenza; H5N6 influenza; H6N1 influenza; H7 influenza; H7N9 influenza; H9N2 influenza; HCoV-EMC; herpes virus; Human coronavirus 229E (HCoV-229E); Human coronavirus HKU1; Human coronavirus NL63 (HCoV-NL63, New Haven coronavirus); Human coronavirus OC43 (HCoV-OC43); Influenza A; Influenza B; influenza virus; MERS-COV; respiratory syncytial virus (RSV); rhinovirus; SARS-COV; SARS-CoV-1; SARS-COV-2; West Niles Virus; Zika Virus; and a variant thereof.

18. (canceled)

19. The method of claim 2,
wherein at least 90 wt % of the tdsRNA is larger than a size selected from the group consisting of: 40 basepairs; 50 basepairs; 60 basepairs; 70 basepairs; 80 basepairs; and 380 basepairs; or
wherein at least 90 wt % of the tdsRNA is smaller than a size selected from the group consisting of: 50,000 basepairs; 10,000 basepairs; 9000 basepairs; 8000 basepairs; 7000 basepairs; and 450 basepairs.

20. The method of claim 2, wherein n is a number with a value selected from the group consisting of: 40 to 50,000; 40 to 40,000; 50 to 10,000; 60 to 9000; 70 to 8000; 80 to 7000; and 380 to 450.

21. The method of claim 2,
wherein n is from 40 to 40,000;
wherein the tdsRNA has about 4 to about 4000 helical turns of duplexed RNA strands; or
wherein the tdsRNA has a molecular weight selected from the group consisting of: 2 kDa to 30,000 kDa; 25 kDa to 2500 kDa; and 250 kDa to 320 kDa.

22. The method of claim 2, wherein the tdsRNA comprises
rIₙ·r(C₁₁₋₁₄U)ₙ; and
rugged dsRNA.

23. The method of claim 2,
wherein
the rugged dsRNA has a molecular weight of about 250 kDa to 500 kDa;
each strand of the rugged dsRNA is from about 400 to 800 basepairs in length;
or
the rugged tdsRNA has about 30 to 100 or 30-60 helical turns of duplexed RNA.

24. The method of claim 2, wherein administering is at least one selected from the group consisting of:
intranasal administration; inhalation administration; intravenus administration;
systemic administration; and topical administration.

25. The method of claim 2, wherein administering tdsRNA is performed by a delivery system or medical device.

26. The method of claim 5, wherein the delivery system or medical device is at least one selected from the group consisting of:
a nebulizer; a sprayer; a nasal pump; a squeeze bottle; a nasal spray; a syringe sprayer; a plunger sprayer; a swab; a pipette; a nasal irrigation device; and a nasal rinse.

27. The method of claim 2,
wherein the tdsRNA is administered at an intranasal dosage of about 0.1 μg to 1,200 μg; 0.1 to 25 μg; 25 μg to 50 μg; 50 μg to 100 μg; 100 μg to 200 μg; 200 μg to 400 μg; 400 μg to 800 μg; 800 μg to 1,250 μg; 1250 μg to 1500 μg; 1500 μg to 2000 μg; or 2000 μg to 2500 μg; or
wherein the tdsRNA is administered at a dosage of 25 mg to 700 mg of tdsRNA per day; 20 mg to 200 mg of tdsRNA per day; 50 mg to 150 mg of tdsRNA per day; or 80 mg to 140 mg of tdsRNA per day; or
wherein the tdsRNA is administered
two times a week at 200 mg per administration, or
wherein the tdsRNA is administered
two times a week at 200 mg per administration for the first 2 weeks, or
two times a week at 400 mg per administration after the first 2 weeks.

28. The method of claim 2, wherein the tdsRNA is administered at a frequency selected from the group consisting of:
one dose per day; one dose every 2 days; one dose every 3 days; one dose every 4 days; one dose every 5 days, one dose a week, two doses a week, three doses a week, one dose every two weeks, one dose every 3 weeks, one dose every 4 weeks, and one dose a month.

29. The method of claim 2, wherein the method is combined with a second treatment for the viral infection.

30. The method of claim 2, wherein the second treatment is an intravenous administration of tdsRNA.

31. A method for treating a viral infection in a subject comprising nasally administering tdsRNA to the subject during nasal viral replication,
wherein nasally administering is at least two or more nasal administrations during nasal virus replication in the subject;
wherein treating is at least one selected from the group consisting of: reducing nasal virus protein level; reducing nasal virus nucleic acid level; reducing nasal peak viral load; reducing nasal time to peak viral load; reducing nasal duration of viral shedding; reducing nasal viral load area under the curve (viral AUC);
and reducing nasal virus titer;
wherein the method enhances cross protection; enhances epitope spreading;
enhances cross reactivity; or enhances mucosal immunity;
in the subject.

32. The method of claim 31, wherein nasal administration is continued at least every other day until nasal virus protein level or nasal virus levels is reduced by at least 90%.

33. The method of claim 31, wherein the method
enhances cross protection against a second virus;
enhances epitope spreading to epitopes in a second virus;
enhances cross reactivity against a second virus; or enhances mucosal immunity against a second virus.

34-36. (canceled)
